(19) United States
(12) Patent Application Publication
(10) Pub. No.: US 2019/0365314 A1
(43) Pub. Date: Dec. 5, 2019

(54) OCULAR FUNDUS IMAGE PROCESSING DEVICE AND NON-TRANSITORY COMPUTER-READABLE MEDIUM STORING COMPUTER-READABLE INSTRUCTIONS

(71) Applicant: NIDEK CO., LTD., Gamagori-shi (JP)

(72) Inventors: Ryosuke SHIBA, Gamagori-shi (JP); Yoshiki KUMAGAI, Toyokawa-shi (JP); Yusuke SAKASHITA, Okazaki-shi (JP)

(73) Assignee: NIDEK CO., LTD., Gamagori-shi (JP)

(21) Appl. No.: 16/427,446

(22) Filed: May 31, 2019

(30) Foreign Application Priority Data
Jun. 4, 2018 (JP) 2018-107281

(51) Int. Cl. A61B 5/00; A61B 3/12; A61B 3/14; A61B 5/107; A61B 3/00

(52) U.S. Cl. CPC A61B 5/489; A61B 3/12; A61B 3/0025; A61B 5/7267; A61B 5/1075; A61B 3/14

(57) ABSTRACT

An ocular fundus image processing device includes a processor. The processor acquires an ocular fundus image of a subject's eye photographed using an ocular fundus image photographing unit. The processor also acquires a detection result of an artery and a vein in at least a part of the ocular fundus image, by inputting at least the part of the ocular fundus image into a mathematical model trained using a machine learning algorithm.

### CROSS REFERENCE TO RELATED APPLICATION

This application is based upon and claims the benefit of priority of Japanese Patent Application No. 2018-107281 filed on Jun. 4, 2018, the contents of which are incorporated herein by reference in its entirety.

### BACKGROUND

The present disclosure relates to an ocular fundus image processing device that processes an ocular fundus image of a subject's eye, and a non-transitory computer-readable medium storing computer-readable instructions.

By observing an ocular fundus, it is possible to ascertain a state of an artery and a vein in a non-invasive manner. In related art, a detection result of an artery and a vein (hereinafter sometimes collectively referred to as “an artery/vein”) obtained from an ocular fundus image is used in various diagnostics and the like. For example, a known ocular fundus image processing device detects an artery/vein by performing image processing on an ocular fundus image. More specifically, the ocular fundus image processing device detects a blood vessel. In the ocular fundus, by calculating, with respect to each of the pixels in the ocular fundus image, a luminance value difference between the pixel and surrounding pixels. Next, the ocular Hindus image processing device uses at least one of the luminance or a diameter of a pixel configuring the detected blood vessel to determine whether the blood vessel is an artery or a vein.

### SUMMARY

Embodiments of the broad principles derived herein provide an ocular fundus image processing device capable of appropriately acquiring a detection result of an artery and a vein in an ocular fundus image, and a non-transitory computer-readable medium storing computer-readable instructions.

Embodiments provide an ocular fundus image processing device that includes a processor. The processor acquires an ocular fundus image of a subject's eye photographed using an ocular fundus image photographing unit, and the processor acquires a detection result of an artery and a vein in at least a part of the ocular fundus image, by inputting at least the part of the ocular fundus image into a mathematical model trained using a machine learning algorithm.

Embodiments further provide a non-transitory computer-readable medium storing computer-readable instructions that, when executed by a processor of an ocular fundus image processing device, cause the ocular fundus image processing device to perform processes including: acquiring an ocular fundus image of a subject's eye photographed using an ocular fundus image photographing unit; and acquiring a detection result of an artery and a vein in at least a part of the ocular fundus image, by inputting at least the part of the ocular fundus image into a mathematical model trained using a machine learning algorithm.

### BRIEF DESCRIPTION OF THE DRAWINGS

FIG. 1 is a block diagram showing an overall configuration of an ocular fundus image processing system 100.

FIG. 2 is a flowchart of ocular fundus image processing.

FIG. 3 is a diagram showing an example of an ocular fundus image 20 in which a region of interest 25 is set.

FIG. 4 is an explanatory diagram illustrating an example of a training data set 30.

### DETAILED DESCRIPTION

As one example, when image processing is used on an ocular fundus image to detect an artery/vein, various issues may arise. For example, it may be difficult to detect an artery/vein using the image processing, such as when an artery and a vein intersect each other, when the ocular fundus image is dark due to an influence of a cataract or the like, when disease is present in the ocular fundus, and the like.

As another example, when detecting an artery/vein from a wide region of the ocular fundus image, it may be difficult to reduce a processing amount, and time may be required to perform the detection. In particular, when processing is performed on each of pixels, the processing amount may easily reach an enormous amount.

The present disclosure provides an ocular fundus image processing device capable of resolving at least one of these problems and appropriately acquiring a detection result of an artery and a vein in an ocular fundus image, and a non-transitory computer-readable medium storing computer-readable instructions.

A processor of an ocular fundus image processing device disclosed in the present disclosure acquires an ocular fundus image photographed using an ocular fundus image photographing unit. The processor acquires a detection result of an artery and a vein in at least a part of the ocular fundus image, by inputting at least the part of the ocular fundus image into a mathematical model trained using a machine learning algorithm. In this case, for example, by training the mathematical model using at least one of the ocular fundus image in which the artery and the vein intersect each other, the ocular fundus image of insufficient brightness due to the influence of a cataract or the like, the ocular fundus image in which disease is present, or the like, as the training data, the detection result of the artery/vein can be appropriately obtained, even with respect to various ocular fundus images.

As the ocular fundus image input into the mathematical model, an image photographed by a variety of ocular fundus image photographing units may be used. For example, at least one of an image photographed using a fundus camera, an image photographed using a scanning laser ophthalmoscope (SLO), an image photographed using an OCT device, or the like may be input into the mathematical model.

The mathematical model may be trained using an ocular fundus image of the subject's eye previously photographed as input training data, and using data indicating an artery and a vein in the ocular fundus image of the input training data as output training data. The detection result of the artery and the vein may be acquired by inputting the one ocular fundus image into the mathematical model. In this case, for example, the artery and the vein can be appropriately detected using simple processing, in comparison to a case using a method in which, after detecting blood vessels, the detected blood vessels are classified into the artery and the vein, a case using a method in which a plurality of sections extracted from the one ocular fundus image are each input into the mathematical model, and the like.

A format of the input training data and the output training data used to train the mathematical model may be selected as appropriate. For example, the color ocular fundus image of the subject's eye photographed using the fundus camera may be used as the input training data. The ocular fundus image of the subject's eye photographed using the SLO may be used as the input training data. The output training data may be generated by an operator specifying, on the ocular fundus image, positions of the artery and the vein in the ocular fundus image of the input training data (by assigning a label indicating the artery and a label indicating the vein on the ocular fundus image, for example).

The processor may set a region of interest inside a part of a region of the acquired ocular fundus image. The processor may acquire a detection result of an artery and a vein in the region of interest by inputting an image of the region of interest into the mathematical model. In this case, the detection result of the artery and the vein can be acquired with less arithmetic processing, compared to a case in which the entire acquired ocular fundus image is input into the mathematical model.

The processor may set, as a region of interest, a region centering on a papilla, inside a region of the ocular fundus image. A plurality of blood vessels, including an artery and a vein, enter into and leave the papilla. Thus, by setting the region centering on the papilla as the region of interest, appropriate information about the artery and the vein (information about the diameter of each of the artery and the vein, for example) can be efficiently acquired. In detection processing of an artery/vein using conventional image processing, since broad-view information over a wide range of the ocular fundus image (position relationships of various sections, for example) is required, it is difficult to accurately detect the artery/vein using only the information for the region of interest. In contrast to this, when using the mathematical model, since the detection processing is performed on the basis of local regions centering on individual pixels, detection processing of a high efficiency using the image of the region of interest can be performed.

The region of interest may be changed. For example, the region of interest according to the present disclosure is an annular region centering on the papilla. However, the shape of the region of interest may be a shape other than the annular shape (a circular shape, a rectangular shape, or the like, for example). The position of the region of interest may be changed. For example, the region of interest may be set centering on the fovea centralis. More specifically, by setting the region of interest centering on the fovea centralis, a blood vessel density around a non-perfusion area of the fovea centralis, or the like, may he calculated. In this case, by using an OCT angiography image (an OCT motion contrast image, for example) as the ocular fundus image, the blood vessel density can be more accurately calculated.

The processor may set at least one of the position or the size of the region of interest in relation to the ocular fundus image, in accordance with a command input by a user. In this case, the user can set the region of interest as the user desires. The processor may detect a specified position of the ocular fundus (the papilla, for example) by performing image processing or the like on the ocular fundus image, and may automatically set the region of interest on the basis of the detected specified position. The processor may detect the size of a specified section of the ocular fundus and may determine the size of the region of interest on the basis of the detected size. For example, the processor may detect the diameter of the papilla that is substantially circular, and may determine, as the diameter of the region of interest, a diameter that is N times the detected diameter of the papilla (N may be set as desired, and may be “3” or the like, for example). A mathematical model may be used that is trained using an ocular fundus image of a subject's eye previously photographed as input training data, and using data indicating the specified position (the position of the papilla, for example) or a specified region (the annular region centering on the papilla, for example) in the ocular fundus image of the input training data as output training data. In this case, the region of interest may be set by inputting the ocular fundus image into the mathematical model.

The processor may calculate data relating to at least one of the detected artery or vein, based on the detection result. In this case, the user can perform a more favorable diagnosis and the like. The data to be calculated may be changed as appropriate. For example, at least one of an average value and a standard deviation of the diameter of the artery, an average value and a standard deviation of the diameter of the vein, an average value and a standard deviation of the diameter of the blood vessel including the artery and the vein, an average value and a standard deviation of a luminance of the artery, an average value and a standard deviation of a luminance of the vein, and an average value and a standard deviation of a luminance of the blood vessel including the artery and the vein, or the like may be calculated as data of the blood vessel.

The processor may calculate a ratio of the diameter of the detected artery and the diameter of the detected vein (hereinafter referred to as an “artery-vein diameter ratio”). In this case, by referring to the artery-vein diameter ratio, the user can more appropriately perform various diagnoses, such as arteriosclerosis and the like.

System Configuration

Hereinafter, an exemplary embodiment of the present disclosure will be described with reference to the drawings. As an example, in a present embodiment, a personal computer (hereinafter referred to as a “PC”) 1 acquires data of an ocular fundus image of a subject's eye (hereinafter referred to simply as an “ocular fundus image”) from an ocular fundus image photographing device 11, and performs various types of processing on the acquired ophthalmic image. In other words, in the present embodiment, the PC 1 functions as an ocular fundus image processing device. However, a device that functions as the ocular fund us image processing device is not limited to the PC 1. For example, the ocular fundus image photographing device 11 may function as the ocular fundus image processing device. A tablet terminal or a mobile terminal, such as a smartphone and the like, may function as the ocular fundus image processing device. A server capable of acquiring the ocular fundus image from the ocular fundus image photographing device 11 via a network may function as the ocular fundus image processing device. Processors of a plurality of devices (a CPU 3 of the PC 1 and a CPU 13 of the ocular fundus image photographing device 11, for example) may perform the various types of image processing in concert with each other.

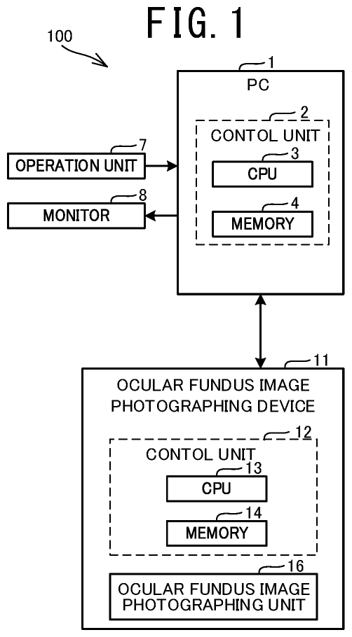

As shown in FIG. 1, an ocular fundus image processing system 100 exemplified by the present embodiment includes the PC 1 and the ocular fundus image photographing device 11. The PC 1 includes a control unit 2 that performs various types of control processing. The control unit 2 includes the CPU 3 and a memory 4. The CPU 3 is a controller that performs control. The memory 4 can store programs, data, and the like. The memory 4 stores an ocular fundus image processing program that is used to perform ocular fundus image processing to be described below. The PC 1 is connected to an operation unit 7 and a monitor 8. The operation unit 7 is operated for the user to input various commands into the PC 1. The operation unit 7 may use at least one of a keyboard, a mouse, a touch panel, or the like, for example. A microphone or the like that is used to input the various commands may be used along with the operation unit 7 or in place of the operation unit 7. The monitor 8 is an example of a display, which can display various images.

The PC 1 can perform reception and transmission of various types of data (the data of the ocular fundus image, for example) with the ocular fundus image photographing device 11. A method for the PC 1 to perform the reception and transmission of the data with the ocular fundus image photographing device 11 may be selected as appropriate. For example, the PC 1 may perform the reception and transmission of the data with the ocular fundus image photographing device 11 using at least one of wired communication, wireless communication, or a detachable storage medium (a USB memory, for example).

Various devices that photograph an image of the ocular fundus of the subject's eye may be used as the ocular fundus image photographing device 11. For example, the ocular fundus image photographing device 11 used in the present embodiment is a fundus camera that can photograph a color image of the ocular fundus using visible light. Thus, processing for detecting an artery/vein (to be described below) can be appropriately performed on the basis of the color ocular fundus image. However, a device other than the fundus camera (at least one of an OCT device, a scanning laser ophthalmoscope (SLO), or the like, for example) may be used. The ocular fundus image may be a two-dimensional front image of the ocular fundus photographed from the front side of the subject's eye, or may be a three-dimensional image of the ocular fundus.

The ocular fundus image photographing device 11 includes a control unit 12, which performs various types of control processing, and an ocular fundus image photographing unit 16. The control unit 12 includes the CPU 13 and a memory 14. The CPU 13 is a controller that performs control. The memory 14 can store programs, data, and the like. The ocular fundus image photographing unit 16 includes optical members and the like to photograph the ocular fundus image of the subject's eye.

Ocular Fundus Image Processing

Hereinafter, the ocular fundus image processing of the present embodiment will be explained in detail. In the ocular fundus image processing of the present embodiment, a detection result of an artery and a vein in the ocular fundus image are acquired, using a mathematical model trained using a machine learning algorithm. The ocular fundus image processing is performed by the CPU 3 in accordance with the ocular fundus image processing program stored in the memory 4.

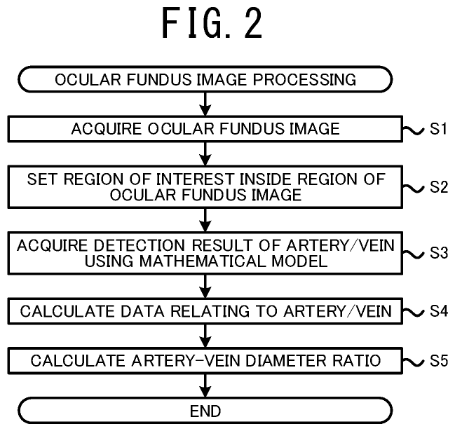

As shown in FIG. 2, when the ocular fundus image processing is started, the CPU 3 acquires the ocular fundus image of the subject's eye (step S1). In the present embodiment, the CPU 3 acquires, from the ocular fundus image photographing device 11, the ocular fundus image (the color front image of the ocular fundus in the present embodiment) photographed by the ocular fundus image photographing unit 16 of the ocular fundus image photographing device 11. A method of acquiring the ocular fundus image may be changed as appropriate. For example, when the ocular fundus image photographing device 11 performs the ocular fundus image processing, the CPU 13 of the ocular fundus image photographing device 11 may acquire the ocular fundus image stored in the memory 14.

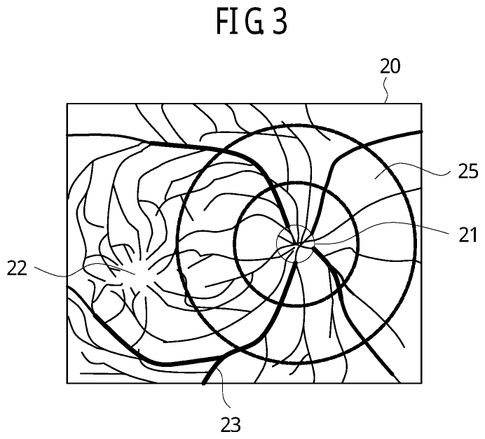

Next, the CPU 3 sets a region of interest in a section inside a region of the ocular fund us image acquired at step S1 (step S2). FIG. 3 shows an example of an ocular fundus image 20 in which a region of interest 25 is set. As shown in FIG. 3, the ocular fundus image 20 of the present embodiment is the color front image of the ocular fundus. An optic papilla (hereinafter referred to as the “papilla”) 21, a macula lutea 22, and an ocular fundus blood vessel 23 of the subject's eye are displayed in the ocular fundus image 20 of the present embodiment. In the present embodiment, inside the region of the ocular fundus image 20, the CPU 3 sets a region centering on the papilla 21 (more specifically, an annular region centering on the papilla 21) as the region of interest 25. The region of interest 25 exemplified in FIG. 3 is a region surrounded by two concentric circles shown by dotted lines. The arteries and the veins enter into and leave the papilla 21. Thus, by setting the region centering on the papilla 21 as the region of interest 25, appropriate information about the arteries and the veins can be efficiently acquired.

The CPU 3 detects the position of the papilla 21 by performing image processing on the ocular fundus image 20, and automatically sets the region of interest 25 on the basis of the detected position of the papilla 21. However, a specific method for setting the region of interest 25 may be changed. For example, the CPU 3 may automatically set the region of interest 25 using a mathematical model that is trained using a machine learning algorithm. The position of the region of interest 25 may be changed. For example, the region of interest 25 may be set having the fovea centralis (the center of the macula lutea 22) as the center of the region of interest 25. The shape of the region of interest 25 may be changed. In the present embodiment, the size of the region of interest 25 is set in advance. However, the CPU 3 may determine the size of the region of interest 25 on the basis of a size of a specified portion in the ocular fundus. For example, the CPU 3 may detect the diameter of the papilla 21 that is substantially circular, and may determine, as a diameter of the region of interest 25, a diameter that is N times (three times, for example) the detected diameter. A value of N may be set in advance, or may be set in accordance with a command input by the user. In this case, the size of the region of interest 25 can be appropriately determined in accordance with the size of the specified portion (the papilla 21 in the present embodiment). Further, the CPU 3 may set at least one of the position or the size of the region of interest 25 in the ocular fundus image 20 in accordance with a command input via the operation unit 7 or the like by the user.

Next, the CPU 3 acquires a detection result of an artery and a vein (step S3), by inputting at least a part (an image of the region of interest 25 in the present embodiment) of the ocular fundus image 20 into the mathematical model trained using the machine learning algorithm. A method of acquiring the detection result of the artery/vein in the present embodiment will be explained in detail. As the machine learning algorithm, for example, a neural network, random forest, boosting, a support vector machine (SVM), and the like are generally known.

The neural network is a technique that imitates the behavior of a nerve cell network of a living organism. Examples of the neural network include, for example, a feedforward neural network, a radial basis function (RBF) network, a spiking neural network, a convolutional neural network, a recurrent neural network (a recurrent neural network, a feedback neural network, and the like), a probabilistic neural network (a Boltzmann machine, a Bayesian network, and the like), and so on.

The random forest is a method to generate multiple decision trees, by performing learning on the basis of training data that is randomly sampled. When the random forest is used, branches of a plurality of decision trees learned in advance as discriminators are followed, and an average (or a majority) of results obtained from each of the decision trees is taken.

The boosting is a method to generate strong discriminators by combining a plurality of weak discriminators. By causing sequential learning of simple and weak discriminators, strong discriminators are constructed.

The SVM is a method to configure two-class pattern discriminators using linear input elements. For example, the SVM learns linear input element parameters from training data, using a reference (a hyperplane separation theorem) that calculates a maximum margin hyperplane at which a distance from each of data points is maximum.

In the present embodiment, a multi-layer neural network is used as the machine learning algorithm. The neural network includes an input layer used to input data, an output layer used to generate data to be predicted, and one or more hidden layers between the input layer and the output layer. A plurality of nodes (also known as units) are arranged in each of the layers. More specifically, a convolutional neural network (CNN) that is a type of the multi-layer neural network is used in the present embodiment.

The mathematical model indicates, for example, a data structure for predicting a relationship between input data and output data. The mathematical model is constructed as a result of training using a training data set. The training data set is a set of input training data and output training data. When a piece of input training data is input, the mathematical model is trained to output the output training data corresponding to the input data. For example, as a result of the training, correlation data (weighting, for example) between the inputs and outputs is updated.

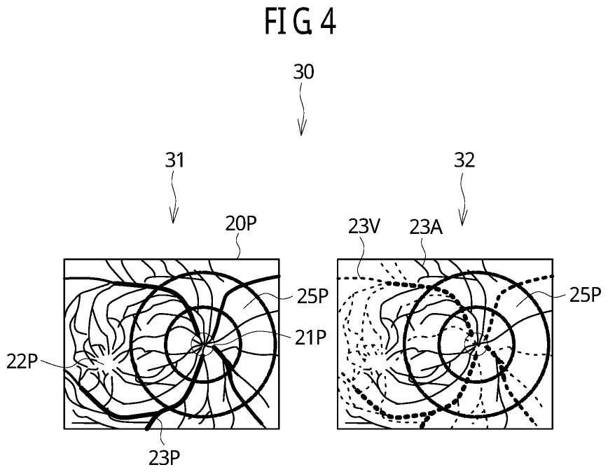

A training data set 30 used to construct the mathematical model of the present embodiment will be explained with reference to FIG. 4. In the present embodiment, a plurality of ocular fundus images 20P of the subject's eye previously captured are used as input training data 31. Further, data indicating arteries 23A and veins 23V in the ocular fundus images 20P of the input training data 31 are used as output training data 32. In the present embodiment, the output training data 32 are generated by an operator assigning a label indicating an artery 23A and a label indicating a vein 23V, on the ocular fundus image 20P of the input training data 31. In the example shown in FIG. 4, the arteries 23A are indicated by solid lines and the veins 23V are indicated by dotted lines. In the example shown in FIG. 4, the input training data 31 indicates the ocular fundus image 20P that is larger than a region of interest 25P. Further, the output training data 32 indicate the arteries 23A and the veins 23V outside the region of interest 25P, in addition to the arteries 23A and the veins 23V inside the region of interest 25P. Thus, for example, even if the position of the region of interest 25 set in the ocular fundus image 20 is changed or the like, the artery/vein can be appropriately detected. However, the input training data 31 may indicate an image of the region of interest 25P, and the output training data 32 may indicate the arteries/veins inside the region of interest 25P.

A plurality of the training data sets 30 include data of a section in which an artery and a vein intersect each other. Thus, by using the constructed mathematical model, a detection result of the artery/vein can be appropriately obtained even in the section in which the artery and the vein intersect each other. Further, the plurality of training data sets 30 also include the training data set 30 for the ocular fundus image 20 that is insufficiently bright due to an influence of a cataract or the like, and the training data set 30 for the ocular fundus image 20P in which disease or the like is present. As a result, the detection result of the artery/vein can be appropriately obtained even in the case of the dark ocular fundus image 20, or the ocular fundus image 20 in which the disease is present.

The CPU 3 inputs at least a part (the image of the region of interest 25 in the present embodiment) of the ocular fundus image 20 (refer to FIG. 3) into the constructed mathematical model. As a result of this, the detection result of the artery/vein included in the input image is output. In the present embodiment, each of pixels of the input image is classified into one of three categories of organism, namely, “artery,” “vein,” or “other,” in this way, the detection result of the artery/vein is output. Specifically, the detection result can be easily obtained by inputting a single image into the mathematical model. Thus, the artery/vein can be appropriately detected using simple processing, compared to a case in which, after detecting the blood vessels, the detected blood vessels are classified into arteries and veins. Further, the processing load can be more easily reduced, compared to a case in which a plurality of sections (patches) extracted from the ocular fundus image are each input into the mathematical model.

As described above, in the present embodiment, the image of the region of interest 25 in the ocular fundus image 20 is input into the mathematical model. Thus, compared to a case in which the entire ocular fundus image 20 is input into the mathematical model, the detection result of the artery/vein can be acquired using less arithmetic processing.

Further, in the present embodiment, when a single ocular fundus image 20 (the image of the region of interest 25) is input into the mathematical model, the arithmetic processing is performed in order from a region of a part of the input image to other regions. The CPU 3 sequentially displays, on the monitor 8, the detection result of the region for which the arithmetic processing is complete. Further, using the detection result of the region for which the arithmetic processing is complete, the CPU 3 sequentially performs data calculation processing (step S4) and artery-vein diameter ratio calculation processing (step S5) to he described below, and sequentially displays the result on the monitor 8. While the processing at step S4 and step S5 is being performed, the arithmetic processing is performed with respect to a remaining region. Thus, the processing can be more efficiently performed. In the present embodiment, the arithmetic processing is performed on the input image, in order from a region of high priority to a region of low priority (in order from the inside of the region of interest 25 toward the outside of the region of interest 25, for example). As a result, the user can ascertain processing a result far the region of high priority at an earlier stage.

Next, the CPU 3 calculates data relating to at least one of the detected artery or vein (step S4). More specifically, in the present embodiment, the CPU 3 calculates an average value and a standard deviation of the diameter of the artery, an average value and a standard deviation of the diameter of the vein, an average value and a standard deviation of the diameter of a blood vessel including the artery and the vein, an average value and a standard deviation of a luminance of the artery, an average value and a standard deviation of a luminance of the vein, and an average value and a standard deviation of a luminance of the blood vessel including the artery and the vein,

Next, the CPU 3 calculates the artery-vein diameter ratio that is a ratio between the diameters of the artery and the vein (step S5). The user can appropriately perform various diagnoses, such as arteriosclerosis and the like, by referring to the artery-vein diameter ratio. A specific method of calculating the artery-vein diameter ratio may be selected as appropriate. For example, the CPU 3 may separately calculate the ratio of the diameters of the arteries and the veins extending upward from the papilla 21, and the ratio of the diameters of the arteries and the veins extending downward from the papilla 21. It is a characteristic of blood vessels of the ocular fundus that arteries and veins tend to extend in parallel each of upward and downward from the papilla 21. Thus, by calculating the artery-vein diameter ratio above and below the papilla 21, respectively, the possibility can be reduced that the artery-vein diameter ratio is not calculated from arteries and veins that do not extend in parallel to each other. As a result of this, a more effective result can be obtained.

The technology exemplified in the above-described embodiment is merely an example. Therefore, the technology exemplified in the above-described embodiment may be changed. First, it is possible to perform only part of the plurality of techniques exemplified in the above-described embodiment. For example, only processing to input the image of the region of interest 25 into a mathematical model may be performed, without using the mathematical model constructed using the method exemplified in the above-described embodiment. In contrast, the detection result of the artery/vein may be acquired using the mathematical model constructed using the method exemplified in the above-described embodiment, without performing the processing to input the image of the region of interest 25 into the mathematical model (namely, by inputting the entire ocular fundus image 20 into the mathematical model).

The apparatus and methods described above with reference to the various embodiments are merely examples. It goes without saying that they are not confined to the depicted embodiments. While various features have been described in conjunction with the examples outlined above, various alternatives, modifications, variations, and/or improvements of those features and/or examples may be possible. Accordingly, the examples, as set forth above, are intended to be illustrative. Various changes may be made without departing from the broad spirit and scope of the underlying principles.

What is claimed is:

1. An ocular fundus image processing device comprising:
a processor, the processor acquiring an ocular fundus image of a subject's eye photographed using an ocular fundus image photographing unit, and the processor acquiring a detection result of an artery and a vein in at least a part of the ocular fundus image, by inputting at least the part of the ocular fundus image into a mathematical model trained using a machine learning algorithm.

2. The ocular fundus image processing device according to claim 1, wherein
the mathematical model is trained using an ocular fundus image of the subject's eye previously photographed as input training data, and using data indicating an artery and a vein in the ocular fund us image of the input training data as output training data, and
the detection result of the artery and the vein is acquired by inputting the one ocular fundus image into the mathematical model.

3. The ocular fundus image processing device according to claim 1, wherein the processor
sets a region of interest inside a part of a region of the acquired ocular fundus image, and
acquires a detection result of an artery and a vein in the region of interest by inputting an image of the region of interest into the mathematical model.

4. The ocular fundus image processing device according to claim 1, wherein
the processor sets, as a region of interest, a region centering on a papilla, inside a region of the ocular fundus image.

5. The ocular fundus image processing device according to claim 1, wherein
the processor calculates data relating to at least one of the detected artery or vein, based on the detection result.

6. The ocular fundus image processing device according to claim 1, wherein
the processor calculates a ratio of a diameter of the detected artery and a diameter of the detected vein.

7. A non-transitory computer-readable medium storing computer-readable instructions that, when executed by a processor of an ocular fundus image processing device, cause the ocular fundus image processing device to perform processes comprising:
acquiring an ocular fundus image of a subject's eye photographed using an ocular fundus image photographing unit; and
acquiring a detection result of an artery and a vein in at least a part of the ocular fundus image, by inputting at least the part of the ocular fundus image into a mathematical model trained using a machine learning algorithm.
